(19) United States
(12) Patent Application Publication
(10) Pub. No.: US 2025/0027928 A1
(43) Pub. Date: Jan. 23, 2025

(54) OPTICALLY TRANSPARENT MICROELECTRODE ARRAYS FOR ELECTROCHEMICAL AND ELECTROPHYSIOLOGICAL MEASUREMENTS OR STIMULATION

(71) Applicant: FEPOD OY LTD, Helsinki (FI)

(72) Inventors: Tomi LAURILA, Helsinki (FI); Samuel RANTATARO, Helsinki (FI); Niklas WESTER, Helsinki (FI)

(73) Assignee: FEPOD OY LTD, Helsinki (FI)

(21) Appl. No.: 18/712,679

(22) PCT Filed: Apr. 3, 2023
(86) PCT No.: PCT/EP2023/058619

§ 371 Date: May 22, 2024

(30) Foreign Application Priority Data
Apr. 1, 2022 (FI) 20227044

(51) Int. Cl. G01N 33/483; C12N 13/00; G01N 21/64

(52) U.S. Cl. CPC G01N 33/4836; C12N 13/00; G01N 21/6458

(57) ABSTRACT

The present invention relates to a microelectrode array device and method for biological imaging comprising a well for containing a liquid medium; an optically transparent substrate; and a microelectrode array comprising multiple electrode pads. Each electrode pad comprises a conductive film having a thickness between 5 nm and 1000 nm comprising a carbon nanotube network and/or graphene. The microelectrode array further comprises at least one counter electrode and electrically conductive traces connecting the electrode pads with a voltage source and connecting the at least one counter electrode with the voltage source such that a potential difference can be provided between the electrode pads and the counter electrode.

The present invention relates to a microelectrode array device for biological imaging, and a cellular imaging method.

### BACKGROUND

The plasma membranes of living cells contain ion channels, whose opening and closing can adjust the potential between inner and outer plasma membrane surfaces. This membrane potential can be sensed with electrodes and that information can be used in evaluating the cell's state or activity. Sensing the membrane potential is especially important in studying neurons, while various other cell types also undergo membrane potential changes that can be desired to be measured. In addition to that, the cell's state or activity can also be evaluated based on the release of neurotransmitters, Ca²⁺ ions, or other biomolecules. Because of that, it is desired to measure both electrical signals and the release of biomolecules. Electrical signals are measured with electrodes, while the release of biomolecules is detected either by electrochemical electrodes or fluorescence signals. Because of this, a transparent electrode that can measure both electrical and electrochemical signals is highly needed as it can be used to multiplex electrical signals with biochemical signals.

Currently available microelectrode array structures are often made of non-transparent materials, typically metals (U.S. RE38,323 E), making it impossible to image cells/organoids/tissue before, during, or after the measurement through the disclosed microelectrode array (MEA) structure. When considering neurotransmitter measurements, it would be highly important to visually see the cell or cell parts on the measurement pad, as otherwise one cannot be sure whether there is a synapse or electrically active ion channel region on top of the measurement pad. Non-transparent electrodes and wirings also make it impossible to measure fluorescently-labelled biomolecules, such as proteins, lipids, sugars, or Ca²⁺ ion concentration fluctuation. Thus, it is advantageous for the MEA structure to be transparent for the use of both academia and industries such as the pharmaceutical industry.

Although wirings have earlier been made from transparent materials, to the inventor's knowledge there are no viable transparent pad materials or products available. There are some devices using indium tin oxide or conductive polymers, but their electrical, electrochemical, or optical properties can still be improved.

It is an object of the present invention to provide a microelectrode array device which provides the described improvements over known microelectrode array devices.

### SUMMARY

This object is achieved with the features of the independent claims. Dependent claims refer to preferred embodiments.

The microelectrode array (MEA) device described herein could be used as electrochemical sensors or as an electrophysiological instrument to measure and/or stimulate cells, organoids, or explanted tissue sample such as a brain slice. Because the proposed MEA structure is transparent, the cells, organoids, or tissue sample can be imaged with microscopes or optically stimulated through the MEA structure. This enables using microscopes where either the objectives or excitation source, or both, are placed beneath the MEA. This allows the combinatory use of electrochemical/electrophysiological measurements with fluorescence-based techniques or other techniques that require optical observation of the sample, for example patch clamping.

According to a first aspect, the invention relates to a microelectrode array device and method for biological imaging comprising a well for containing a liquid medium; an optically transparent substrate; and a microelectrode array comprising multiple electrode pads. Each electrode pad comprises a conductive film, optionally having a thickness between 5 nm and 1000 nm, comprising a carbon nanotube network and/or graphene. The microelectrode array further comprises at least one counter electrode and electrically conductive traces connecting the electrode pads with a voltage source and connecting the at least one counter electrode with the voltage source such that a potential difference can be provided between the electrode pads and the counter electrode. Such a microelectrode array device has improved electrical, electrochemical, and/or optical properties. Furthermore, the electrode pads are able to perform both electrical measurements and electrochemical measurements, thus improving the functionality of microelectrode array structures. Furthermore, the porous conductive films can be modified with metal particles or conductive polymers to further improve the conductivity and improve chemical sensor performance.

Optionally the substrate is made of at least one of glass, quartz, transparent polymers, transparent metal oxides, and cellulose film.

Optionally, each electrode pad comprises a surface area of between 1 μm² and 1000 μm², optionally between 1 μm² and 500 μm², optionally between 10 μm² and 100 μm². Alternatively, each electrode pad comprises a surface area of between 1 mm² and 100 mm², optionally between 1 mm² and 10 mm².

Optionally, each electrode pad has an optical transmittance within the wavelength spectrum 250 nm to 900 nm of at least 70%, optionally at least 80%, optionally at least 90%; or wherein each electrode pad has an optical transmittance within the wavelength spectrum 250 nm to 3000 nm of at least 70%, optionally at least 80%, optionally at least 90%. Optionally, these optical transmittances are achieved over at least 50%, optionally at least 70%, optionally over at least 90% of the wavelengths in the above-mentioned wavelength spectrums or for each of the wavelengths in the above-mentioned wavelength spectrums. It some cases, an optical transmittance for certain wavelengths (e.g. wavelengths at which a fluorescence is expected may be sufficient).

In some embodiments, the conductive film is deposited using press transfer, spin coating, dip coating, liquid-phase adsorption, spray coating, inkjet printing, screen printing, electrochemical deposition, arc discharge method, evaporation, sputtering, physical vapor deposition, chemical vapor deposition (CVD), or plasma-enhanced CVD, optionally wherein the film is deposited using aerosol chemical vapor deposition (CVD) dry deposited by press-transfer.

Optionally, the carbon nanotube network film has a thickness of between 10 nm and 400 nm, optionally between 30 nm and 200 nm, optionally between 30 nm and 100 nm.

Optionally, the electrically conductive traces are formed of the same material as the electrode pads. Optionally, the multiple electrode pads are arranged in a grid or repeating pattern.

Optionally, the microelectrode array device further comprises contact pads, positioned on the optically transparent substrate adjacent to the grid of multiple electrode pads, wherein one contact pad exists for each electrode pad; and wherein the electrically conductive traces connect each pair of electrode pad and contact pad.

Optionally, the multiple electrode pads are further coated with cellular growth promoters, optionally wherein the growth promoters include collagen, laminin, polyornitine (Poly-L-Ornithine) and/or extracellular matrix factors.

According to a further aspect, the invention is also directed to a cellular imaging method using the microelectrode array device, the method involving the steps of
i. depositing cells or tissue on the microelectrode array within the well;
ii. measuring the voltage and/or current from each of the electrode pads; and
iii. imaging the cells or tissue using an optical and/or fluorescence microscope.

Optionally, steps ii and iii are performed simultaneously and/or wherein step iii includes fluorescent imaging of fluorescently labeled cells or tissues.

Optionally, the method includes a further step of introducing a biochemical reagent to the well during step ii, optionally wherein the biochemical reagent includes a neurotransmitter, pharmacological agent, and/or cell signal mediator.

Optionally, the method includes a further step of stimulating the cells or tissue by providing a voltage difference between the electrode pads and the at least one counter electrode.

Optionally, step ii includes performing chronoamperometric, voltametric and/or impedimetric measurements of the electrode pads.

### BRIEF DESCRIPTION OF THE DRAWINGS

The subject matter of the invention will be explained in more detail in the following text with reference to preferred exemplary and non-limiting embodiments which are illustrated in the attached drawings. These figures disclose embodiments of the invention for illustrational purposes only. In particular, the disclosure provided by the figures and description is not meant to limit the scope of protection conferred by the invention.

FIG. 1 schematically depicts a top-side view of one example of a microelectrode array design;

FIG. 2 schematically depicts a cross-sectional view of a portion of an example microelectrode array;

FIG. 3 illustrates an example of combined microscopy and electrical and/or electrochemical measurements performed according to the method;

FIG. 4 are exemplary immunofluorescence images of primary cortical astrocytes which are imaged through an optically transparent electrode according to the present invention. The cell was stained for nucleus (DAPI, blue), focal adhesions (vinculin, green), and actin cytoskeleton (phalloidin, red). Commonly used filters (DAPI, FITC, TXRED) were used that provide data derived along the entire fluorescence microscopy wavelength range, and the filters are denoted above the figures.

FIG. 5 is a graph of experimental results of chronoamperometric performance of a transparent CNT network sheet sample, measured during a dopamine injection series (0 nM, 25 nM, 50 nM, 100 nM, 250 nM, 500 nM, 1000 nM dopamine) in cell culturing medium. Each line represents an individual electrode pad;

FIG. 6 is a graph of experimental results of the chronoamperometric performance of another transparent CNT network sheet sample, measured by a dopamine injection series (0 nM, 25 nM, 50 nM, 100 nM, 250 nM, 500 nM, 1000 nM dopamine) in cell culturing medium. Each line represents an individual electrode pad;

FIG. 7 is a graph of experimental results of the transmittance of light through a glass reference slide and a glass reference slide upon which a carbon nanotube network (CNT) as used in the electrode pads according to the present invention has been deposited (lower line); and

FIG. 8 presents two examples of chronoamperometric data from one electrode pad with a high iron (A-C) SWCNT conductive film and a second electrode pad with a low iron (D-F) SWCNT conductive film.

### DETAILED DESCRIPTION

While the invention has been illustrated and described in detail in the drawings and foregoing description, such illustration and description are to be considered illustrative or exemplary and non-restrictive; the invention is thus not limited to the disclosed embodiments. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality and may mean “at least one”.

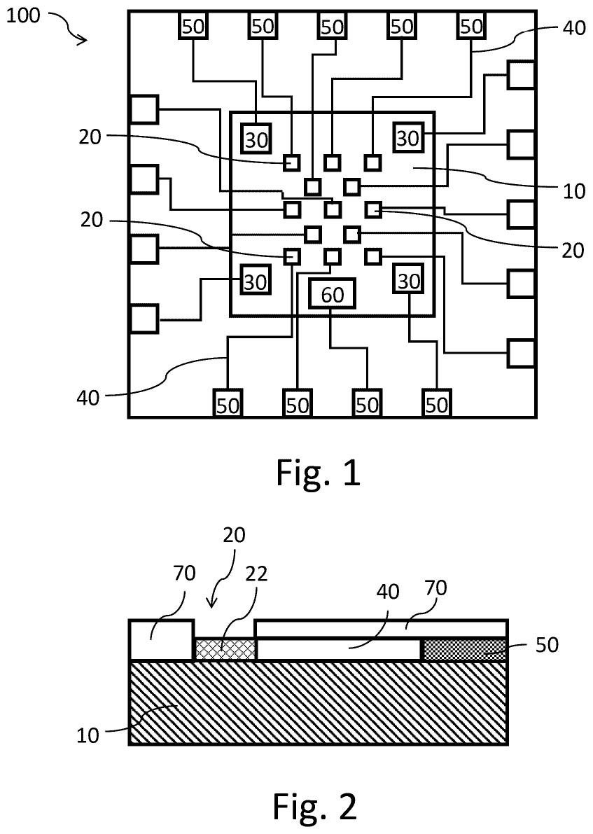

FIG. 1 depicts one example of the microelectrode array 100 which can be implemented within the microelectrode array device. The microelectrode array 100 is formed on an optically transparent substrate 10. The microelectrode array 100 comprises multiple electrode pads 20, which may, as in FIG. 1, be arranged in a grid or patterned array on the substrate 10 surface. The substrate 10 also supports at least one counter electrode 30, which together with the electrode pads 20 can close an electrical circuit in connection with an outside voltage source. Electrically conductive traces 40 are also supported on the substrate 10 which connect the electrode pads 20 with a provided voltage source.

The substrate 10 is made of optically transparent materials including but not limited to glass, quartz, transparent polymers, metal oxides, cellulose film, or a combination of the aforementioned materials. If the substrate 10 is conductive, an insulation material 70, shaped as a layer, can be placed between the substrate 10 and the electrode pad. In some embodiments the substrate 10 forms the entire bottom surface of the well 200. Alternatively, the optically transparent substrate 10 may only support the electrode pads 20 whereas other features, such as the counter electrodes 30 are positioned on an adjacent, non-transparent substrate within the well. In the example shown in FIG. 1, the optically transparent substrate 10 underlies the electrode pads 20, the counter electrodes 30 and any reference electrodes 60, if present (the dashed-line inner square of FIG. 1).

Electrically conductive traces 40 can be formed of the same material as the electrode pads 20. In the case of an electrode pad formed of carbon nanotubes, the CNT network layer can also simultaneously be deposited as the conductive traces 40, and the conductivity of the conductive traces 40 can be improved by the addition of conductive polymers. Alternatively, the conductive traces 40 can be made of transparent materials such as conductive polymers, indium tin oxide (ITO), graphene, graphene nanoparticles, or the combination of aforementioned materials. The electrical signal originating from the electrode pads 20 can be transmitted to an oscilloscope, potentiostat, or any other device that is capable of producing or reading electrical signals including but not limited to potential, current, or frequency.

The electrode pads 20 comprise a conductive film 22 which comprises or is made of a carbon nanotube network (CNT) and/or a graphene layer. In some particular embodiments, the conductive film 22 may be formed from single walled carbon nanotubes (SWCNT). The CNT network and/or graphene can be placed onto the substrate 10 by techniques including but not limited to press transfer, spin coating, dip coating, liquid-phase adsorption, spray coating, inkjet printing, screen printing, electrochemical deposition, arc discharge method, evaporation, sputtering, physical vapor deposition, chemical vapor deposition (CVD), or plasma-enhanced CVD. The conductive film 22 layer can be patterned to a desired surface pattern with top-down methods such as laser patterning, dry etching, oxygen plasma etching, mechanical removal from non-protected areas, wet etching. Alternatively, the conductive film 22 can be directly made into the desired pattern by techniques such as screen printing, inkjet printing, 3D-printing, lift-off, or techniques based on modifying surface chemistries to obtain selective deposition with spin coating, dip coating, spray coating, or liquid-phase adsorption. Advantageously, the microelectrode array 100 structure as described herein can be manufactured on a large scale without necessarily requiring the use of an ultra-clean cleanroom environment and tooling. As such, the microelectrode array 100 structure can be manufactured directly on large substrates 10 and the structure is also capable to be manufactured on a roll-to-roll basis.

In the case of a conductive film 22 which comprises or is made of graphene, optionally chemical vapor deposition (CVD) graphene, the growth and transfer is a commonly known process. Single or multilayer graphene would most conveniently be grown with CVD onto a metal catalyst foil, most commonly copper. After that the graphene side of the foil is coated with a polymer (usually PMMA) or polymer tape and then Cu foil is etched away in an etching bath. The film is fished out/transferred onto a substrate (usually Si/SiO₂) and dipped in washing solution to remove the polymer. After cleaning the film can be further transferred onto other substrates or processed into devices by pattering (eg. laser pattering or lithography+oxygen plasma etching). Such transfer films (for example, trivial transfer graphene available from ACS Material) and transfer films on polymer (Easy Transfer: Monolayer Graphene on Polymer Film available from Graphenea) are commercially available. Alternatively inks with graphene flakes, optionally with conductive polymers, could be ink jet printed.

The conductive film 22 can also be modified with enzymes that modify an analyte of interest into another molecule and a side-product that is detected, for example using the enzyme L-glutamate oxidase to transform L-Glutamate (analyte of interest) into α-ketoglutaric acid+ammonia+H₂O₂ (that is detected electrochemically). Alternatively, the conductive layer can be modified with antibodies or aptamers to bind a specific biomolecule, altering the state of electrode (typically impedance) that can be measured and that information can be used to deduce the concentration of that specific biomolecule. Specific binding of targets/analytes/antigens can also block active area of the electrode or result in less efficient diffusion of electroactive species from the culturing medium (could also be added redox reporters). Conformational changes upon binding of analytes could have same effect. Thus changes in voltammeteric or chronoamperometric signal can be correlated to changes in targets/analytes/antigens concentrations. The conductive film 22 can also be modified with metal films and/or metal-containing nano- and/or microparticles to improve sensitivity and/or selectivity towards a specific analyte of interest, as well as conductivity. Further, the conductive films 22 can be coated with growth promotors which can improve cell viability.

A conductive polymer coating (eg. polypyrrole or PEDOT:PSS) may be added to the conductive film 22. This may help to provide the conductive film 22 with higher surface area, higher electrical conductivity and/or electrochemical activity. The polymer coating would further improve the conductivity by doping, for example, a CNT layer and partially filling any gaps between the CNT nanowires in the film, which may particularly be helpful when wires have very small linewidths. Such a polymer coating may also protect the electrode from biofouling and render it more biocompatible. However, a CNT conductive film 22 alone (with appropriate protein coating) is sufficiently biocompatible and sensitive, even after the fouling that takes place in cell culturing media.

Depending on the needs of the specific experiment being employed, the impedance of the conductive film 22 may be controlled by adjusting the thickness or the density of the network. The diameter of the electrode pad may be easily adapted, for example between 10,000 μm, depending on whether the electrode is desired to be used in measuring individual cells or cell populations. Each electrode pad may provide a surface area of between 1 μm² and 1000 μm², optionally between 1 μm² and 500 μm², optionally between 10 μm² and 100 μm².

The microelectrode array device as described herein is advantageous because it allows to multiplex information obtained from the electrical or electrochemical recordings and/or stimulation with fluorescence microscopy techniques. The transparent microelectrode array device described herein includes or is made of optically transparent materials, with the potential exception of external contact pads 50, making it possible to image cells or organoids or tissue samples before and during the electrical or electrochemical measurements of the same cells/organoids/tissues. Furthermore, the same electrode pads 20 can perform electrophysiological measurement techniques and electrochemical techniques, thus providing multiple functionalities of the same electrode pads 20.

The microelectrode array device as described herein enables automated or non-automated continuous or non-continuous electrochemical/electrical/optical detection or stimulation for cells, organoids, or tissue samples. This device can also enable high through-put evaluation of the electronic state of cells or organoids or tissue sample, potentially including a given any chemical substance to the cells or organoids or tissue sample.

Optionally, the microelectrode array 100 may further comprise one or more reference electrodes 60. The signal from the reference electrode 60 may be used to remove noise from the electrode pad signals. Alternatively, a reference electrode 60 may be provided as an external Pt, Ag or Ag/AgCl wire or Ag/AgCl reference electrode in a glass capillary. The reference electrode is used in electrochemistry (such as in a three-electrode cell with wiring, counter and reference) to sense the potential of the working electrode to accurately apply the desired potential. A reference electrode provides a stable well-known potential.

FIG. 2 depicts a cross-sectional side view of a portion of the microelectrode array 100. While the features are not shown to scale, it is evident that an optically transparent substrate 10 supports the electrode pads 20, which are connected through the conductive traces 40 to the external contact pads 50. In between electrode pads 20 and potentially also covering conductive traces 40 insulation material 70 may be provided. This can be advantageous for ensuring that electric signals do not dissipate through the conductive traces 40 or travel along undesired pathways in between electrode pads 20. It may also help to obtain spatial resolution in the microelectrode array so as to be able to isolate a cell/organoid or population of cells at a cite of interest that is observed with the microscope. The insulation material helps to electrically isolate the conductive traces 40 from the culture medium with a dialectic coating. This insulation material 70 could be provided with screen-printable polymers comprising materials based on siloxanes or phenoxyphenylsilane, UV-curable epoxy-based polymers such as SU-8, or liquid glass or spin-on-glass. These materials may also be applied by alternative coating techniques such as inkjet printing, 3D-printing, spin coating, dip coating, or traditional UV or electron beam or micro- or nanoimprint lithography techniques.

Alternative designs of the microelectrode array 100 are envisioned, as the wiring could be positioned slightly above or below the measurement pad area. Optionally, the external contact pads 50 can be positioned slightly above or below the wiring. The contact pads 50 are optionally made at the edges of the microelectrode array 100 and/or substrate 10. These contact pads 50 can be made of either the aforementioned transparent materials or alternatively, the use of non-transparent conductive materials such as metals is also possible if the contact pads 50 are not within the imaging field of view.

Conductive traces 40 can be insulated with an insulator material that is transparent in the optical and fluorescence microscopy wavelength regime (250-900 nm), but not necessarily limited to this wavelength regime. The insulator material may also contain absorption peaks within the 250-900 nm wavelength range if it does not interfere with fluorescence microscopy. The MEA device may be insulated with screen-printable materials including but not limited to those based on siloxanes or phenoxyphenylsilane, UV-curable epoxy-based polymers such as SU-8, liquid glass, or spin-on-glass. The insulator material(s) may also be applied by alternative coating techniques such as inkjet printing, 3D-printing, spin coating, dip coating, physical vapor deposition, chemical vapor deposition, atomic layer deposition, or traditional UV or electron beam or micro-/nanoimprint lithography techniques. If alternative coating techniques are used, commonly used insulator materials may be used, including but not limited to silicon dioxide, silicon nitride, metal oxides such as aluminum oxide or titanium oxide, or transparent polymers that are not autofluorescent in the wavelength range 250-800 nm, such as parylene C.

FIG. 3 is an illustration of one potential use of the present invention. The microelectrode array device includes a substrate 10 upon which the microelectrode array 100 is situated. The device comprises a well 200, which is adapted to contain a liquid medium on top of the substrate 10 and the microelectrode array 100. The device may be placed on an optical and/or fluorescent microscope. Simultaneously the contact pads 50 of the array are connected to a voltage source 300, potentiostat and/or oscilloscope, which can provide a current through the electrode pads 20. Due to the optical transparency of the electrode pads 20 any cells or tissue present within the well 200 can be imaged at the same time as the cells/tissues are electrically stimulated through the electrode pads 20. Alternatively or additionally, the electrode pads 20 may measure electrical activity generated by cells or tissue through the connection of the contact pads 50 with a potentiostat and/or oscilloscope.

The contact pads 50 can be connected to an external voltage source 300 as well as a computer and/or external control system. The control system can be implemented to provide an appropriate potential, current, frequency, pulse width, etc. to the electrode pads 20. Simultaneously, the electrode pads 20 can be monitored for changes in impedance and potential resultant from cellular activity.

One potential embodiment of the microelectrode array device is employing an optically transparent microelectrode array 100 for measuring action potentials in combination with high temporal/low temporal resolution combined with electrochemical analysis. For this implementation the action potential is typically around 1 kHz with low impedance required. Recommended electrode diameters for single-cell are in the range of 10 to 1000 μm, optionally 100 μm to 1000 μm. In this embodiment a carbon nanotube network can be used as both the conductive film 22 of the electrode pad and the conductive traces 40. Optionally, the electrode pad may also comprise additional metal and/or conductive polymer coatings.

In another embodiment, large diameter electrode pads 20 (comprises a surface area of between 1 mm² and 100 mm², optionally between 1 mm² and 10 mm²) may be employed having low impedance, which can be fabricated for monitoring of network cellular or tissue phenomena in combination with electrical activity of single cells. For example, studying voltage-gated sodium channels of cancer cells requires the ability to measure orders of magnitudes lower amplitudes. The small electrodes of commercially available microelectrode arrays 100 have too high impedances for such measurements. However, a microelectrode array device according to the present invention can measure the signal from voltage-gated sodium channels effectively.

In other embodiments, the microelectrode array device may be shaped as a neural probe, and then the electrode pad locations are positioned along the probe. Further, optogenetic modulation of cells, organoids, or tissue samples can potentially be performed on the microelectrode array device. Optogenetic modulation of ion channels or proteins can be performed wherein the effect can be directly measured with the measurement pad. This could be used in e.g. studying how neurotransmitter vesicles fuse with the plasma membrane, in addition to many other applications. Additionally, electrochemoluminescence cell imaging is also enabled due to electrochemical activity of the electrode pad and its transparency. In further embodiments the inventive microelectrode array device could be combined with a microfluidic device.

In one example of the embodiment, single walled carbon nanotube (SWCNT) networks were fabricated in a laminar flow reactor by chemical vapor deposition and were collected using a membrane filter. The SWCNT network sample was press-transferred onto a transparent polymer substrate, after which the network was densified by adding 99.9% ethanol (obtained from Anora, Finland) and letting it evaporate in air. Electrical contact was made by adding conductive silver paste (obtained from Electrolube, United Kingdom) and drying overnight in air, after which a conductive copper tape (obtained from Ted Pella, USA) was used to contact the silver paste to copper slide. An inert PTFE-tape (obtained from Irpola, Finland or alternatively, CHR 2255 Modified High-Modulus PTFE Film Coated with High-Temperature Silicone Adhesive from Saint-Gobain, France) was used to insulate the electrode from other regions. Prior to using the electrode pads, their surfaces were treated by electrochemical oxidation at 1.5 V (vs. Ag/AgCl) potential for 30 seconds in Dulbecco's Phosphate Buffered Saline (PBS, obtained from Gibco, ThermoFisher Scientific, USA), followed by a second treatment at 0.4 V (vs. Ag/AgCl) for 60 seconds to stabilize the surface.

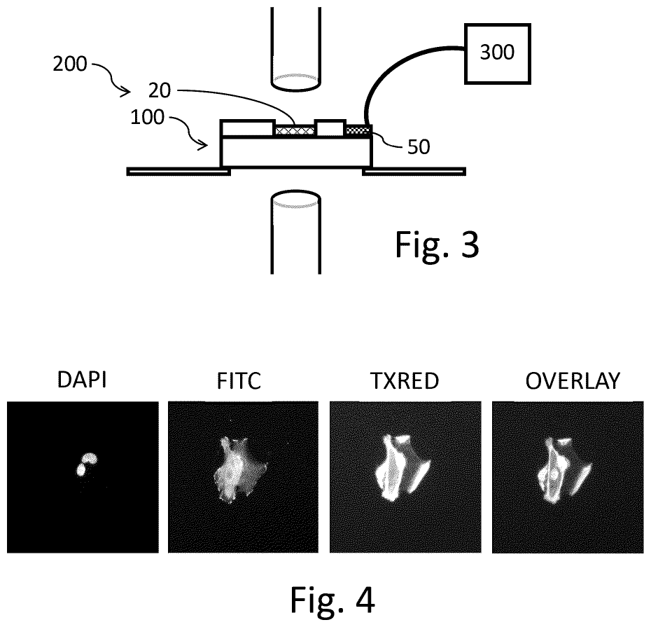

FIG. 4 provides an actual example of the inventive microelectrode device in use. Therein, immunofluorescence images of primary cortical astrocytes are provided which are adhered on top of the microelectrode array 100. The imaging is performed through the electrode. In this example the electrode pads 20 are formed by a CNT network electrode material. The electrode surface was not treated at all to improve cell adhesion or viability, highlighting the excellent biocompatibility of the material as such. The cell was stained for nucleus (DAPI, blue), focal adhesions (vinculin, green), and actin cytoskeleton (phalloidin, red). Commonly used imaging filters (DAPI, FITC, TXRED) were chosen that represent the entire fluorescence microscopy wavelength range. The filters used are labeled above each image.

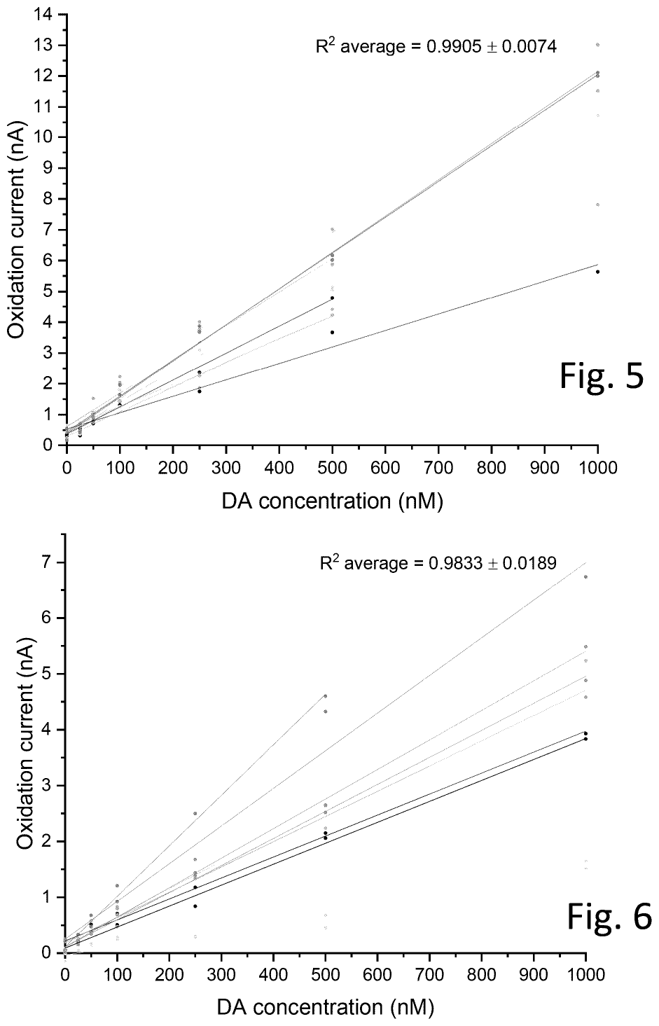

FIG. 5 provides experimental evidence for the excellent chronoamperometric performance of the electrode pads. In this example the electrode pads are formed from a transparent CNT network sheet sample. Current was measured during a dopamine injection series (0 nM, 25 nM, 50 nM, 100 nM, 250 nM, 500 nM, 1000 nM dopamine) in cell culturing medium. Each line represents an individual electrode pad. As is evident therefrom, the electrode pads respond highly linearly to stimulus (R²=0.9905±0.0074) within this physiologically relevant dopamine concentration range.

FIG. 6 provides further experimental evidence for the chronoamperometric performance of the electrode pads. This example utilizes another example of transparent CNT network sheet sample. Oxidation current was measured during a dopamine injection series (0 nM, 25 nM, 50 nM, 100 nM, 250 nM, 500 nM, 1000 nM dopamine) in cell culturing medium. The average individual electrode performs highly linearly (R²=0.9833±0.0189) in this physiologically relevant dopamine concentration range.

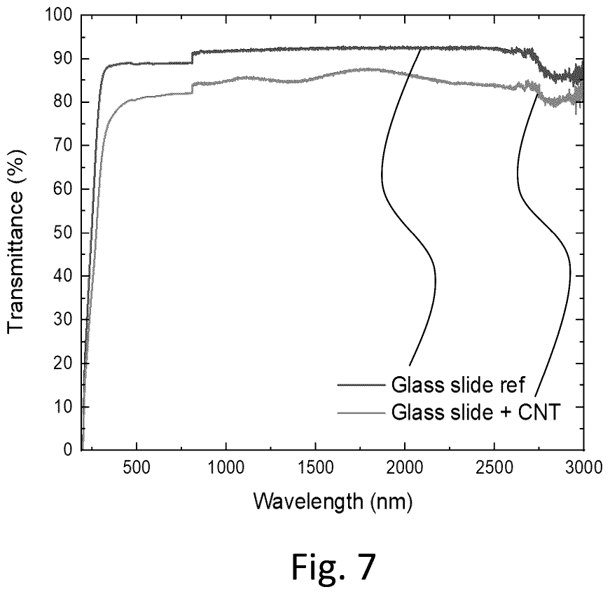

FIG. 7 is a graph of experimental results of the transmittance of light through a glass reference slide and a glass reference side upon which a carbon nanotube network (CNT) as used in the electrode pads according to the present invention has been deposited (lower line) using press-transfer. UV-VIS-NIR spectroscopy measurements were performed of the carbon nanotube network on glass slide compared to glass slide reference. Measurements were conducted though full stack and absorbance includes all materials in the stack. As can be taken therefrom, electrode pads according to the present invention maintain a very high transmittance across the wavelength spectrum from 250 nm up to 3000 nm. Light transmittance within this range is consistently above 70% and is generally around 80%. As such, electrodes pads as described herein are highly advantageous for optical imaging purposes.

A UV-vis spectroscope (Agilent Cary 5000 UV-vis-NIR spectrometer from Agilent Technologies, Inc., United States) was used to characterize absorbance of the SWCNT materials. Transmittance was calculated according to the relation between absorbance and transmittance based on the Beer-Lambert law, as follows:

$$A = - {log}_{10} \frac{{I}_{T}}{{I}_{0}} A = - {log}_{10} T T = {10}^{- A} T ( \% ) = 100 * T$$

where A denotes measured absorbance, T denotes transmittance through the material stack containing glass coverslip substrate (DWK Life Sciences) with SWCNT sheet electrode, Iₒ denotes intensity measured with bare glass coverslip, and Iₜ denotes intensity measured through the sample material.

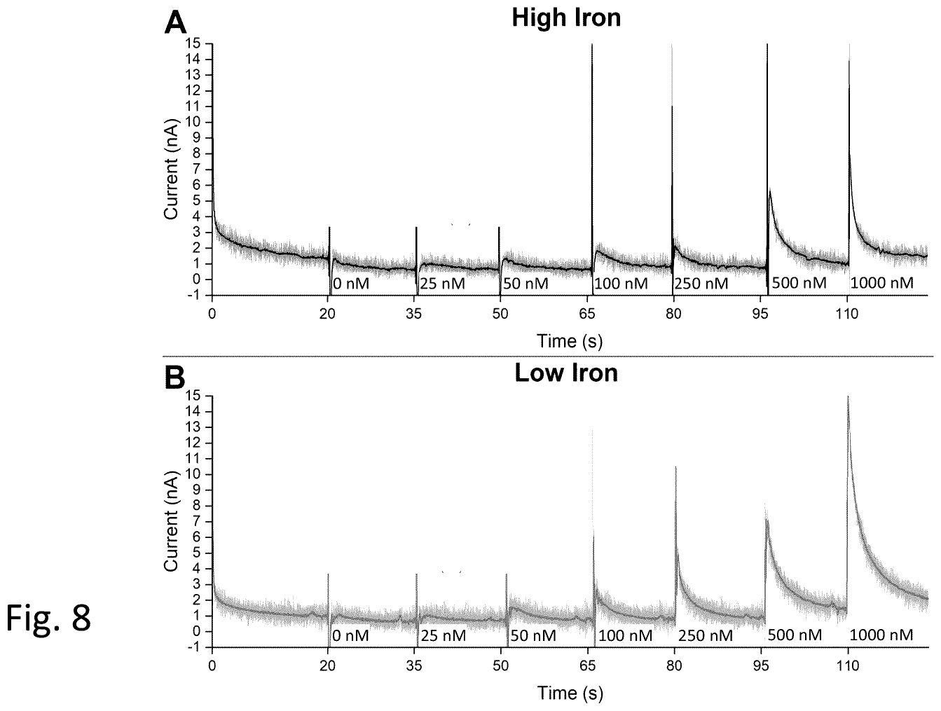

FIG. 8 presents two examples of chronoamperometric data from one electrode pad with high iron (A-C) SWCNT conductive film and a second electrode pad with low iron (D-F) SWCNT conductive film. Each spike in the graph corresponds to the addition of dopamine to the medium as described in connection with FIG. 6. Both types of electrode pads exhibit robust and time-resolved signaling. The high iron and low iron samples were fabricated at different collection rates, 100% and 500%, by modifying the amount of ferrocene available in the gas phase. The amount of iron can be controlled by variating the ferrocene cartridge temperature as it will change the vapor pressure of ferrocene. The SWCNT network synthesized with slower collection rate of 100% is referred to as low iron SWCNT, whereas the sample with collection rate of 500% is referred as high iron SWCNT. In microelectrode arrays of the prior art it is often exceedingly difficult to obtain these kinds of sensitivities in the presence of culturing media due to the fouling of electrodes. Most electrodes that perform at this level in buffers become very rapidly inactivated in buffer medium. Electrode pads according to the present invention, however, do not suffer from this issue.

1.-15. (canceled)

16. A microelectrode array device for biological imaging comprising:
a well for containing a liquid medium;
an optically transparent substrate;
a microelectrode array comprising:
multiple electrode pads, each comprising a conductive film having a thickness between 5 nm and 1000 nm, the conductive film comprising one or both of a carbon nanotube network and graphene;
at least one counter electrode;
electrically conductive traces connecting the electrode pads with a voltage source and connecting the at least one counter electrode with the voltage source such that a potential difference can be provided between the electrode pads and the counter electrode.

17. The device of claim 16, wherein the optically transparent substrate is made of at least one of glass, quartz, transparent polymers, transparent metal oxides, and cellulose film.

18. The device of claim 16, wherein each electrode pad comprises a surface area of between 1 μm² and 1000 μm².

19. The device of claim 16, wherein each electrode pad comprises a surface area of between 1 mm² and 100 mm².

20. The device of claim 16, wherein each electrode pad has an optical transmittance within the wavelength spectrum 250 nm to 900 nm of at least 70%.

21. The device of claim 16, wherein the conductive film is deposited using press transfer, spin coating, dip coating, liquid-phase adsorption, spray coating, inkjet printing, screen printing, electrochemical deposition, arc discharge method, evaporation, sputtering, physical vapor deposition, chemical vapor deposition (CVD), or plasma-enhanced CVD.

22. The device of claim 16, wherein the conductive film comprises a carbon nanotube network film with a thickness of between 10 nm and 400 nm.

23. The device of claim 16, wherein the electrically conductive traces are formed of the same material as the multiple electrode pads.

24. The device of claim 16, wherein the multiple electrode pads are arranged in a grid or repeating pattern.

25. The device of claim 24, wherein the microelectrode array device further comprises contact pads, positioned on the optically transparent substrate adjacent to the grid of multiple electrode pads, wherein one contact pad exists for each electrode pad; and wherein the electrically conductive traces connect each pair of electrode pad and contact pad.

26. The device of claim 16, wherein the multiple electrode pads are further coated with cellular growth promoters.

27. The device of claim 27, wherein the cellular growth promoters include one or more of the following: collagen, laminin, Poly-L-Ornithine, and extracellular matrix factors.

28. The device of claim 16, wherein the conductive film is deposited using aerosol chemical vapor deposition (CVD) dry deposited by press-transfer.

29. The device of claim 16, wherein each electrode pad has an optical transmittance within the wavelength spectrum 250 nm to 900 nm of at least 90%.

30. The device of claim 16, wherein each electrode pad comprises a surface area of between 1 mm² and 10 mm².

31. The device of claim 16, wherein each electrode pad comprises a surface area of between 10 μm² and 100 μm².

32. The device of claim 16, wherein the conductive film comprises a carbon nanotube network film with a thickness of between 30 nm and 200 nm.

33. The device of claim 16, wherein each electrode pad has an optical transmittance within the wavelength spectrum 250 nm to 3000 nm of at least 70%.

34. A cellular imaging method comprising:
providing a microelectrode array device with:
a well for containing a liquid medium;
an optically transparent substrate; and
a microelectrode array comprising:
multiple electrode pads, each comprising a conductive film having a thickness between 5 nm and 1000 nm, the conductive film comprising one or both of a carbon nanotube network and graphene;
at least one counter electrode;
electrically conductive traces connecting the electrode pads with a voltage source and connecting the at least one counter electrode with the voltage source such that a potential difference can be provided between the electrode pads and the counter electrode;
depositing cells or tissue on the microelectrode array within the well;
measuring one or both of the voltage and current from each of the electrode pads; and
imaging the cells or tissue using one or both of an optical and fluorescence microscope.

35. The cellular imaging method of claim 34, wherein the measuring and imaging are performed simultaneously.

36. The cellular imaging method of claim 34, including a further step of introducing a biochemical reagent to the well during the measuring.

37. The cellular imaging method of claim 36, wherein the biochemical reagent includes one or more of the following: a neurotransmitter, a pharmacological agent, and a cell signal mediator.

38. The cellular imaging method of claim 34, including a further step of stimulating the cells or tissue by providing a voltage difference between the electrode pads and the at least one counter electrode.

39. The cellular imaging method of claim 34, wherein the imaging includes fluorescent imaging of fluorescently labeled cells or tissues.

40. The cellular imaging method of claim 34, wherein measuring includes performing one or more of the following measurements of the electrode pads: chronoamperometric, voltametric and impedimetric.
